(19) United States
(12) Patent Application Publication
(10) Pub. No.: US 2012/0259203 A1
(43) Pub. Date: Oct. 11, 2012

(54) Sheath Retractable Flexible Injection Needle

(76) Inventors: Paul David Devereux, Dublin (IE); Shay Joseph Lavelle, Annacotty (IE); Therese Elizabeth Fitzgibbon, Newcastle West (IE); Ciarán Toomey, Rathcormac (IE)

(21) Appl. No.: 13/441,316

(22) Filed: Apr. 6, 2012

Related U.S. Application Data
(60) Provisional application No. 61/473,454, filed on Apr. 8, 2011.

(51) Int. Cl. A61B 1/018; A61M 5/32; A61B 5/055; A61B 6/00; A61B 8/00

(52) U.S. Cl. 600/414; 600/106; 600/424

(57) ABSTRACT

Devices that provide an accurate needle injection depth by way of a sheath covered flexible injection needle that prevents accidental piercing, permits easy unsheathing and re-sheathing of the needle, and permits tactile feedback and direct or indirect visualization to confirm proper needle injection depth are illustrated.

### CROSS-REFERENCE TO RELATED APPLICATIONS

This application claims priority under to U.S. Provisional Application No. 61/473,454, filed on Apr. 8, 2011, the entirety of which is hereby fully incorporated by reference herein.

### TECHNICAL FIELD

The present invention relates to medical devices and more specifically, needles.

### BACKGROUND

Numerous medical procedures require the use of a sheathed needle, that when unsheathed, is used to pierce tissue at a certain depth for delivery of a fluid. For example, Botulinum toxin (trade name Botox®) may be injected into a bladder wall to treat an over-active bladder. Other treatments using sheath-covered needles include those in the field of urology, such as vesicoureteral reflux (VUR) as well as those in the field of endoscopy such as injection into the gastrointestinal mucosa.

The needle is sheathed as it is moved to the injection location to protect the patient, endoscope, cystoscope, or other medical device from accidental piercing whilst the needle is being positioned over the target injection site. The sheath is then retracted exposing a portion of the needle. The user cannot visualize the needle during use to ensure correct placement and injection depth.

### BRIEF SUMMARY

In a first aspect, a medical device for use with an endoscope is provided, the medical device including a needle including a proximal portion and a distal portion, the distal portion of the needle including a sharpened tip; a sheath including a proximal portion, a distal portion, and a lumen extending between the proximal portion and the distal portion of the sheath, wherein at least a portion of the needle is movably disposed through the lumen; and a handle including: an outer handle component fixedly connected to the proximal portion of the needle; an inner handle component fixedly connected to the proximal portion of the sheath, the inner handle component being slidably engaged with the outer handle component, wherein the inner handle component includes a locking pin disposed thereonto; and a plurality of channels disposed in the outer handle component and configured for providing a pathway for axial and transverse movement of the locking pin, the plurality of channels including a longitudinal channel along which the locking pin moves as the sheath is moved relative to the needle to expose or conceal a distal portion of the needle, and a first transverse channel in communication with and disposed at an angle acute to the longitudinal channel; wherein the medical device is configured to be advanced through a working channel of an endoscope; wherein the needle and the sheath each are sufficiently flexible such that angulation of the endoscope is not unduly diminished or hindered when disposed through the working channel of the endoscope.

In a second aspect, a medical device is provided including a needle including a proximal portion and a distal portion, the distal portion of the needle including a sharpened tip; a sheath including a proximal portion, a distal portion, and a lumen extending between the proximal portion and the distal portion of the sheath, wherein at least a portion of the needle is movably disposed through the lumen; and a handle including: an outer handle component fixedly connected to the proximal portion of the needle; an inner handle component fixedly connected to the proximal portion of the sheath, the inner handle component being slidably engaged with the outer handle component, wherein the inner handle component and the outer handle component include complimentary means for guiding or locking a position of the sheath relative to the needle; and an adjustment mechanism attached to the outer handle component, wherein the adjustment mechanism includes a screw to rotatably lengthen and shorten the medical device relative to an endoscope; wherein the medical device is configured to be advanced through a working channel of the endoscope; wherein the needle and the sheath each are sufficiently flexible such that angulation of the endoscope is not unduly diminished or hindered when disposed through the working channel of the endoscope.

In a third aspect, a method of using a sheath retractable flexible injection needle is providing including providing a sheath retractable flexible injection needle having: a needle; a sheath; and a handle including: an outer handle component fixedly connected to the proximal portion of the needle; an inner handle component fixedly connected to the proximal portion of the sheath, the inner handle component being slidably engaged with the outer handle component, wherein the inner handle component includes a locking pin disposed thereonto; and a plurality of channels disposed in the outer handle component and configured for providing a pathway for axial and transverse movement of the locking pin, the plurality of channels including a longitudinal channel along which the locking pin moves as the sheath is moved relative to the needle to expose or conceal a distal portion of the needle, and a first transverse channel in communication with and disposed at an angle acute to the longitudinal channel; wherein the sheath retractable flexible injection needle is configured to be advanced through a working channel of an endoscope; wherein the needle and the sheath each are sufficiently flexible such that angulation of the endoscope is not unduly diminished or hindered when disposed through the working channel of the endoscope; attaching a syringe to a portion of the handle adapted for receiving a syringe; positioning the needle over an injection site; retracting the sheath thereby exposing a portion of the needle; locking the sheath; pushing the needle into the injection site; and injecting a substance into the injection site.

### BRIEF DESCRIPTION OF THE SEVERAL VIEWS OF THE DRAWINGS

The embodiments will be further described in connection with the attached drawing figures. It is intended that the drawings included as a part of this specification be illustrative of the exemplary embodiments and should in no way be considered as a limitation on the scope of the invention. Indeed, the present disclosure specifically contemplates other embodiments not illustrated but intended to be included in the claims.

FIG. 1 illustrates a partial cross-sectional side view of an exemplary sheath retractable flexible injection needle;

FIG. 2 illustrates an exploded view of the device illustrated in FIG. 1;

FIG. 3 illustrates a partial cross-sectional side view of an exemplary sheath retractable flexible injection needle;

FIG. 4 illustrates an exemplary flexible injection needle attached to a working channel of an exemplary endoscope;

FIG. 5 illustrates an exemplary flexible injection needle attached to a working channel of an exemplary endoscope wherein the exemplary flexible injection needle includes an adjustable length mechanism;

FIG. 6 illustrates a sheath adjustor; and

FIG. 7 illustrates a method for using a sheath retractable flexible injection needle.

### DETAILED DESCRIPTION OF PRESENTLY PREFERRED EMBODIMENTS

The exemplary embodiments illustrated herein provide exemplary apparatuses for controlling the extension of a needle from a protective sheath and providing direct visualization of needle depth penetration. The present invention is not limited to those embodiments described herein, but rather, the disclosure includes all equivalents. Moreover, the embodiments illustrated herein can be used in the fields of urology and gastrointestinal endoscopy as well as any other field, and they are not limited to the size or shapes illustrated herein. Indeed, the devices can be used in any field where control of the movement of components relative to each other is desired and can be sized, manufactured, altered, or changed for the particular treatment needed.

Throughout, patient is not limited to being a human being, indeed animals and others are contemplated. User is contemplated throughout the disclosure as being anyone or thing capable of using the device, including but not limited to, a human being and machine.

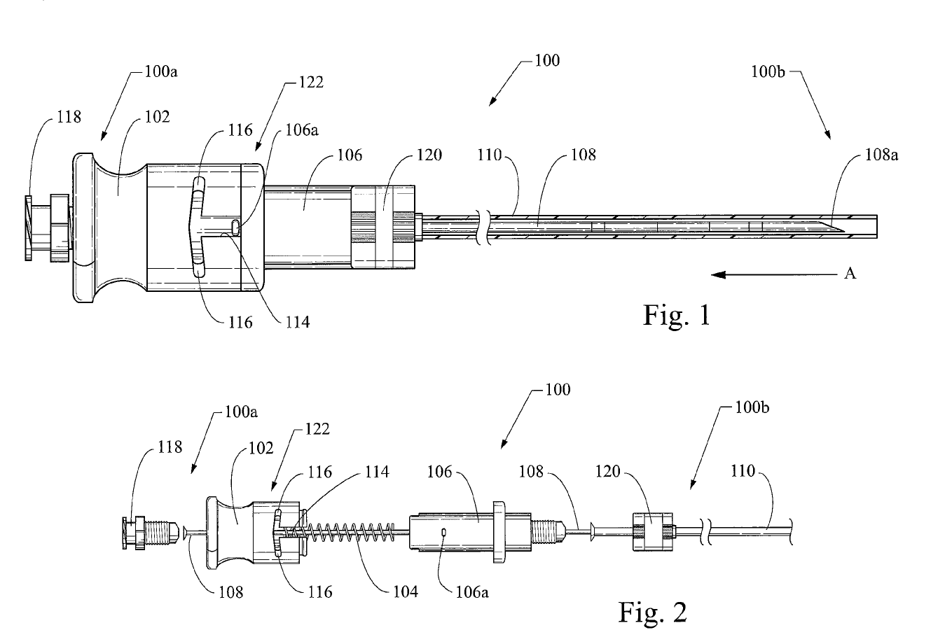

A more detailed description of the embodiments will now be given with reference to FIGS. 1-7. Throughout the disclosure, like reference numerals and letters refer to like elements. The present disclosure is not limited to the embodiments illustrated; to the contrary, the present disclosure specifically contemplates other embodiments not illustrated but intended to be included in the claims.

FIG. 1 illustrates a partial cross-sectional side view of an exemplary sheath retractable flexible injection needle 100, and FIG. 2 illustrates an exploded view of the device 100 illustrated in FIG. 1. Referring to FIGS. 1-2, device 100 has proximal portion 100a and distal portion 100b. An optional syringe attaches to an adaptive connection, such as female luer lock adapter (FLLA) 118, connecting to handle 122, which connects to an adaptive connection, such as male luer lock adapter (MLLA) 120. FLLA 118 and MLLA 120 are manufactured using plastic injection molding, but other manufacturing techniques are contemplated as are forming MLLA and/or FLLA from other medical grade materials, including but not limited to, stainless steel.

FLLA 118 is connected to outer handle component 102 by way of a thread lock, although other methods for connecting are contemplated, including but not limited to adhesive, over molding, and ultrasonic welding. Outer handle component 102 connects to inner handle component 106 by way of a snap-fit connection; although other connection means are contemplated, including but not limited to, adhesive, over molding, thread lock, and ultrasonic welding. Locking pin 106a, disposed on inner handle component 106, moves along channel 114 and engages outer handle component 102 and sheath 110 such that sheath retracts and extends over needle 108 by pulling or pushing inner handle component 106 respective to outer handle component 102. Other means for guiding and locking the sheath position relative to the needle, besides the use of a locking pin and/or channel are contemplated, including put not limited to, a button, spring, and a trigger. Indeed, the arrangement of locking pin 106a and channel 114 could be reversed such that locking pin 106a could be disposed on outer handle component 102 and channel 114 could be disposed on inner handle component 106.

Inner handle component 106 is attached to MLLA 120 which connects to sheath 110. It is contemplated, although not required that MLLA 120 can be used to attach device 100 to another device, such as the working channel of an endoscope, such that device 100 would be locked into place by attachment to an FLLA and thus free a user's hand.

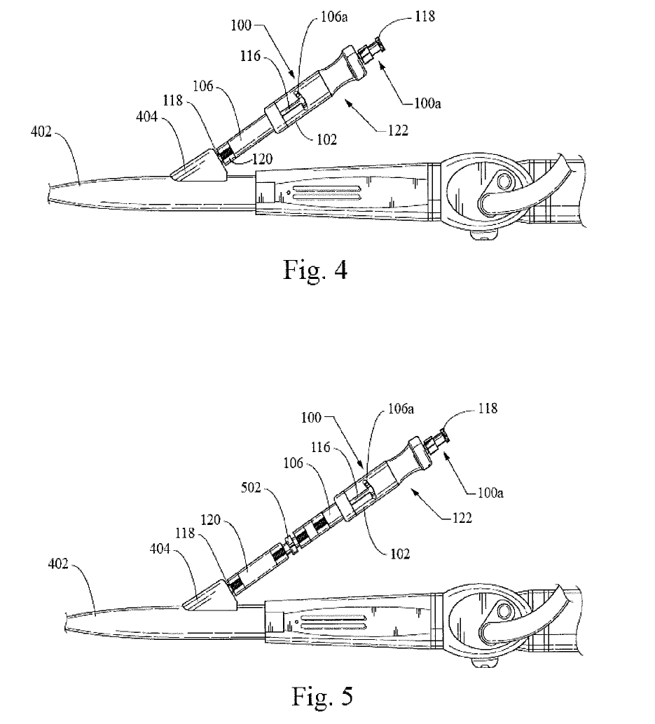

For example, FIG. 4 illustrates exemplary flexible injection needle 100 attached to working channel 404 of exemplary endoscope 402. Configuring flexible injection needle 100 for attachment to another device, such as working channel 404 of endoscope 402 provides many benefits, including but not limited to, providing for a more stable and secure flexible injection needle 100, eliminating the need for an assistant to hold a portion of flexible injection needle 100, improved and easier injections because flexible injection needle 100 is stabilized, and reduction of accidental advancement or retraction of flexible injection needle 100. It is contemplated that flexible injection needle 100 could be manufactured in different lengths to accommodate varying lengths of endoscopes.

FIG. 5 illustrates exemplary flexible injection needle 100 attached to working channel 404 of exemplary endoscope 402, wherein flexible injection needle 100 includes adjustable length mechanism 502 configured for adjusting the length of flexible injection needle 100 by way of a screw, that when rotated about the central axis in a clockwise direction decreases the length of flexible injection needle 100, and when rotated about the central axis in a counter-clockwise direction, it increases the length of flexible injection needle 100. Such a configuration provides many benefits, including but not limited to, permitting a single flexible injection needle 100 for use with endoscopes of varying length by providing adjustment means, such as adjustment mechanism 502, to vary the length of flexible injection needle 100.

Other adjustment means are contemplated. For example, FIG. 6 illustrates a sheath adjustor having sheath 602 and thumb screw 604 for engaging and disengaging a device, to permit the axial movement of sheath to vary the length of a device, the length of which is visualized via measurement markings 606.

Referring again to FIGS. 1-2, sheath 110 is biased in a distal direction due to spring 104, thus covering needle tip 108a. Spring 104 is made from stainless steel; however it is contemplated that it can be made from any material capable of being formed into a spring. The portion of needle 108 in the proximal-most direction is flared and sandwiched between FLLA 118 and outer handle component 102, although other methods for attachment are contemplated, including but not limited to, screwing, gluing, and crimping it. The portion of sheath 110 in the proximal-most direction is flared and sandwiched between inner handle component 106 and MLLA 120, although other methods for attachment are contemplated, including but not limited to, screwing, gluing, and over molding.

Handle components are manufactured using plastic injection molding, although other methods are contemplated as are other materials. The overall length of device 100 is about 40 cm if configured for attachment to a cystoscope; however, other dimensions are contemplated, including but not limited to 70 cm for use with an endoscope, depending upon the needs of the patient, the area to be treated, and the method for positioning device 100.

Indeed, other handles are contemplated, including but not limited to, the use of handles having sliders, turn knobs, thumb screws, etc. for axial retraction and extension of the sheath respective to the needle.

Needle 108 is a 23 gauge needle, although other gauges are contemplated depending upon the needs of the patient and the area to be treated. Needle 108 is a stainless steel sharpened tube about one inch long and is bonded to flexible plastic tubing, although flexibility is not required but is helpful for positioning and maintaining the scope in an angulated or deflected state. Accordingly, it is preferred that the device be flexible such that it does not unduly diminish or excessively hinder scope angulation. Other configurations and materials are contemplated depending upon the needs of the patient and the area to be treated. In some embodiments, a portion of the needle 108 may be surrounded by an over-tube to increase the stiffness of the needle 108 to resist deformation of the needle. The over-tube may be affixed to the needle, with adhesive, welding, or other known attachment methods or structures.

It is contemplated that needle tip 108a is machine ground to a desired sharpness for piercing the area to be treated, including but not limited to, skin, muscle, tissue, bone, or combination thereof. Other configurations and materials are contemplated depending upon the needs of the patient and the area to be treated. Needle 108 is contemplated to being of any size and shape suitable for delivering a fluid, solid, material, or other treatment mechanism, and it can be manufactured in whole or in part from plastic, stainless steel, or other suitable medical-grade materials, including but not limited to, echogenic and other materials that may or may not provide for visualization using a direct or indirect visualization device, including but not limited to, fluoroscopy, x-ray, direct endoscopic visualization, ultrasound, or magnetic resonance imaging (MRI).

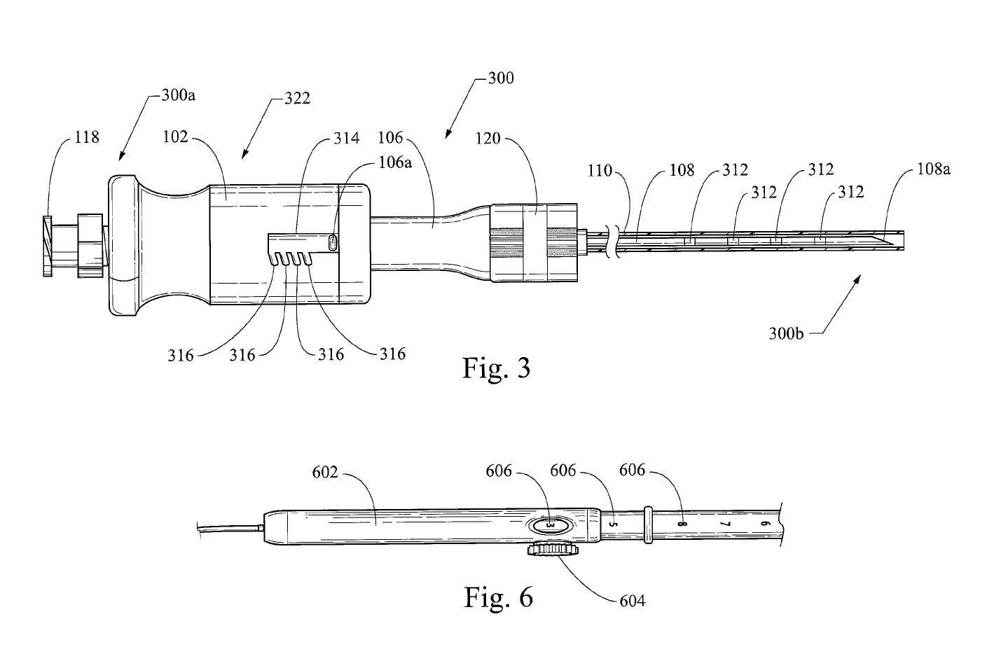

Sheath 110 is retracted in the direction of Arrow A (i.e. the proximal direction) by pulling inner handle component 106 in a proximal direction within channel 114, the position of which is locked into place by moving locking pin 106a into either of transverse channels 116, thus exposing a portion of needle 108, for example about 4 mm. Other dimensions are contemplated, including exposing more or less of needle 108, a variable portion of needle 108, or an incremental portion of needle 108, by providing, for example, additional transverse channels such as those illustrated in FIG. 3.

Transverse channels 116 are disposed at an angle acute to longitudinal channel 114; although other configurations are contemplated including being disposed at an angle perpendicular thereto. Additionally, channels could be further configured with a ridge to provide additional friction to guard against accidental disengagement. Other configurations are contemplated.

To use a sheath retractable flexible injection needle, such as that illustrated in FIGS. 1-2, a user positions needle 108 having sheath 110 extended over needle 108 through a flexible endoscope, cystoscope, or other device or method for positioning a needle over a treatment area. Sheath 110 is retracted by pulling inner handle component 106 in a proximal direction and is locked by moving locking pin 106a into one of transverse channels 116. An optional syringe is attached to FLLA 118, needle 108 is advanced into tissue to the desired depth by having unsheathed needle tip 108a pierce tissue until tissue abuts sheath 110 such that the desired depth of needle 108, such as about 4 mm, is in tissue. Optionally, markers, such as those discussed below, can be utilized on device 100 to provide for direct or indirect visualization of needle 108 using a visualization device, including but not limited to, fluoroscopy, x-ray, direct endoscopic visualization, and MRI. The substance is then injected into the tissue as needed, and the procedure may be repeated in whole or in part depending upon the needs of the patient and the area being treated.

FIG. 3 illustrates a partial cross-sectional side view of an exemplary sheath retractable flexible injection needle 300 having markers 312 and handle 322. Device 300, having a proximal portion 300a and a distal portion 300b, includes channel 314 and a number of transverse channel sheath locks 316 that are spaced at about 2 mm apart, such that sheath 110 exposes about 2 mm, 4 mm, 6 mm, and 8 mm of needle tip 108a when inner handle component 106 is pulled and locking pin 106a is navigated from channel 314 into one of transverse channel locks 316. Other spacing increments are contemplated, including but not limited to, the use of non-uniform increments, such as about 1 mm, 2 mm, 4 mm, and 8 mm. Transverse channel locks 316 are disposed at an angle acute to longitudinal channel 314; although other configurations are contemplated including being disposed at an angle perpendicular. Handle 322 is easy to operate, for example, because control sheath locks 316 are on the same side, but other configurations are contemplated.

Optional markers 312 provide for direct endoscopic visualization, however, it is contemplated that markers can also provide for indirect visualization, such as, for example, through the use of ultrasound. For example, it is contemplated that markers may be made in whole or in part from materials, including but not limited to, stainless steel, radiopaque materials, Platinum-Iridium alloy or echogenic materials, such as gold and tungsten having surface irregularities. An echogenic material includes surface irregularities that reflect ultrasonic waves and thus, allow the material to be seen with ultrasonic imaging devices. Echogenic techniques are described in U.S. Pat. No. 5,081,997 and U.S. Pat. No. 5,289,831 and are hereby incorporated by reference in their entirety. Thus, markers 312 provide for visualization outside the patient using a direct or indirect visualization device, including but not limited to, fluoroscopy, x-ray, direct endoscopic visualization, ultrasound, or MRI.

As illustrated in FIG. 3, optional markers 312 are etched onto the surface of needle 108 and provide for direct endoscopic visualization at increments of, for example, about 2 mm, 4 mm, 6 mm, and 8 mm such that the user knows the depth at which needle tip 108a penetrates the area to be treated, including but not limited to, the wall of the lower urinary tract, using a means other than tactile feedback from when tissue abuts sheath 110. Other distances and dimensions are contemplated based upon the needs of the patient and the area to be treated. Moreover, location of markers 312 is not limited to distal portion 300b of device 300; they may be located elsewhere, in different quantity, or need not be used at all. Additionally, because markers 312 are optionally set apart at a known distance and/or are disposed having known lengths, they can also aid in measuring internal bodily or foreign structures.

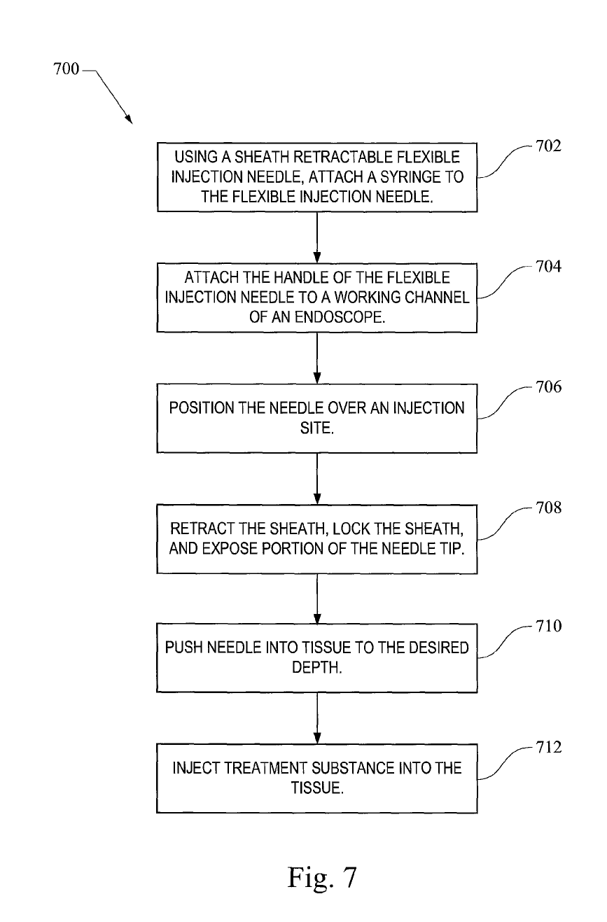

FIG. 7 illustrates a method for using a sheath retractable flexible injection needle 700, such as those illustrated in FIGS. 1-5. At block 702, using a sheath retractable flexible injection needle, such as those illustrated in FIGS. 1-3, a syringe is attached to a connector of the sheath retractable flexible injection needle adapted for receiving a syringe, such as FLLA 118 illustrated in FIGS. 1-3. Alternatively, the syringe may be integral to the device, and the syringe may be connected to the device or removed therefrom at various times throughout.

At block 704, the handle of the flexible injection needle is attached to a working channel of an endoscope. Optionally, the length of the sheath retractable flexible injection needle may be varied to correspond with the length of the endoscope by adjusting an adjustment mechanism, such as that illustrated in FIGS. 5-6.

At block 706, the needle is positioned over an injection site by way of a flexible endoscope, cystoscope, or other means for positioning a needle over a treatment area.

At block 708, the sheath is retracted by pulling an inner handle component of the device, such as inner handle component 106 illustrated in FIGS. 1-3, thereby exposing a desired portion of the needle tip, for example about 4 mm, and the sheath is locked into place.

At block 710, the needle is pushed into the tissue to the desired depth as determined by tactile feedback from, for example, tissue abutting the sheath or by, for example, the visualization of markers, such as markers 312 (illustrated in FIG. 3), using a direct or indirect visualization means, including but not limited to, fluoroscopy, x-ray, direct endoscopic visualization, ultrasound, or MRI.

At block 712, a treatment substance is injected into the patient as needed. Optionally, the needle can be re-sheathed as needed for removal, to alternate positioning, or as treatment needs dictate.

The procedure can be repeated in whole or in part depending upon the needs of the patient and the area to be treated. For example, to treat urge urinary incontinence where an overactive bladder (OAB) contracts spontaneously whilst it is filling, substances such as Botulinum toxin (trade name Botox®), or other materials, may be injected into the wall of the lower urinary tract to bind the nerve endings and the release of the chemicals that cause the bladder to contract suddenly and involuntarily. Multiple injections may be needed in the same or different locations.

From the foregoing, it can be seen that the present disclosure provides devices having an accurate needle injection depth by way of a sheath covered flexible injection needle that prevents accidental piercing, permits easy unsheathing and re-sheathing of the needle, and permits tactile feedback and direct or indirect visualization to confirm proper needle injection depth.

What is claimed is:

1. A medical device for use with an endoscope comprising:
a needle comprising a proximal portion and a distal portion, the distal portion of the needle comprising a sharpened tip;
a sheath comprising a proximal portion, a distal portion, and a lumen extending between the proximal portion and the distal portion of the sheath, wherein at least a portion of the needle is movably disposed through the lumen; and
a handle comprising:
an outer handle component fixedly connected to the proximal portion of the needle;
an inner handle component fixedly connected to the proximal portion of the sheath, the inner handle component being slidably engaged with the outer handle component, wherein the inner handle component comprises a locking pin disposed thereonto; and
a plurality of channels disposed in the outer handle component and configured for providing a pathway for axial and transverse movement of the locking pin, the plurality of channels comprising a longitudinal channel along which the locking pin moves as the sheath is moved relative to the needle to expose or conceal a distal portion of the needle, and a first transverse channel in communication with and disposed at an angle acute to the longitudinal channel;
wherein the medical device is configured to be advanced through a working channel of an endoscope; wherein the needle and the sheath each are sufficiently flexible such that angulation of the endoscope is not unduly diminished or hindered when disposed through the working channel of the endoscope.

2. The medical device of claim 1, further comprising a proximal connector connected to a proximal portion of the handle, wherein the connector is configured for receiving a syringe.

3. The medical device of claim 1, further comprising:
a distal connector connected to a distal portion of the handle and disposed about a portion of the sheath and the needle, wherein the distal connector is configured for attachment to the working channel of the endoscope, and
an adjustment mechanism in communication with the handle, wherein the adjustment mechanism is configured to vary the length of the medical device relative to the endoscope.

4. The medical device of claim 3, wherein the adjustment mechanism further comprises a screw configured to rotate to adjust the length of the medical device.

5. The medical device of claim 3, wherein the adjustment mechanism further comprises a sheath in communication with a screw configured for rotational attachment to the working channel of the endoscope.

6. The medical device of claim 1, wherein the first transverse channel comprises a plurality of transverse channels in communication with and disposed at angles acute to the longitudinal channel at spaced apart locations and configured to lock the sheath in a plurality of incremental positions relative to the needle.

7. The medical device of claim 6, wherein the longitudinal channel is in direct communication with the plurality of transverse channels.

8. The medical device of claim 6, wherein the incremental positions are configured to expose about 2 mm, 4 mm, 6 mm, and 8 mm of the distal portion of the needle.

9. The medical device of claim 1, wherein the needle further comprises at least one marker disposed about the needle, wherein the marker is configured for direct or indirect visualization using a visualization device.

10. The medical device of claim 9, wherein the visualization device is fluoroscopy, x-ray, direct endoscopic visualization, ultrasound, or magnetic resonance imaging (MRI).

11. A medical device comprising:
a needle comprising a proximal portion and a distal portion, the distal portion of the needle comprising a sharpened tip;
a sheath comprising a proximal portion, a distal portion, and a lumen extending between the proximal portion and the distal portion of the sheath, wherein at least a portion of the needle is movably disposed through the lumen; and
a handle comprising:
an outer handle component fixedly connected to the proximal portion of the needle;
an inner handle component fixedly connected to the proximal portion of the sheath, the inner handle component being slidably engaged with the outer handle component, wherein the inner handle component and the outer handle component comprise complimentary means for guiding or locking a position of the sheath relative to the needle; and
an adjustment mechanism attached to the outer handle component, wherein the adjustment mechanism comprises a screw to rotatably lengthen and shorten the medical device relative to an endoscope;
wherein the medical device is configured to be advanced through a working channel of the endoscope; wherein the needle and the sheath each are sufficiently flexible such that angulation of the endoscope is not unduly diminished or hindered when disposed through the working channel of the endoscope.

12. The medical device of claim 11, further comprising a distal connector connected to a distal portion of the handle and disposed about a portion of the sheath and the needle, wherein the distal connector is configured for attachment to the working channel of the endoscope.

13. The medical device of claim 11, wherein the complimentary means for guiding or locking a position of the sheath relative to the needle comprises a plurality of channels disposed in the outer handle component and configured for providing a pathway for axial and transverse movement of a locking pin in communication with the inner handle component, the plurality of channels comprising a longitudinal channel along which the locking pin moves as the sheath is moved relative to the needle to expose or conceal a distal portion of the needle, and a plurality of transverse channels in communication with and disposed at angles acute to the longitudinal channel at spaced apart locations and configured to lock the sheath in a plurality of incremental positions relative to the needle.

14. The medical device of claim 13, wherein the longitudinal channel is in direct communication with the plurality of transverse channels.

15. The medical device of claim 11, wherein the needle further comprises at least one marker disposed about the needle, wherein the marker is configured for direct or indirect visualization using a visualization device.

16. The medical device of claim 11, wherein the complimentary means for guiding or locking a position of the sheath relative to the needle comprises a locking pin and a channel.

17. A method of using a sheath retractable flexible injection needle comprising:
providing a sheath retractable flexible injection needle comprising:
a needle;
a sheath; and
a handle comprising:
an outer handle component fixedly connected to the proximal portion of the needle;
an inner handle component fixedly connected to the proximal portion of the sheath, the inner handle component being slidably engaged with the outer handle component, wherein the inner handle component comprises a locking pin disposed thereonto; and
a plurality of channels disposed in the outer handle component and configured for providing a pathway for axial and transverse movement of the locking pin, the plurality of channels comprising a longitudinal channel along which the locking pin moves as the sheath is moved relative to the needle to expose or conceal a distal portion of the needle, and a first transverse channel in communication with and disposed at an angle acute to the longitudinal channel;
wherein the sheath retractable flexible injection needle is configured to be advanced through a working channel of an endoscope; wherein the needle and the sheath each are sufficiently flexible such that angulation of the endoscope is not unduly diminished or hindered when disposed through the working channel of the endoscope;
attaching a syringe to a portion of the handle adapted for receiving a syringe;
positioning the needle over an injection site;
retracting the sheath thereby exposing a portion of the needle;
locking the sheath;
pushing the needle into the injection site; and
injecting a substance into the injection site.

18. The method of claim 17, wherein the method further comprises attaching the sheath retractable flexible injection needle to the working channel of the endoscope.

19. The method of claim 17, wherein the method further comprises visualizing a portion of the needle using a visualization device.

20. The method of claim 19, wherein the visualization device is fluoroscopy, x-ray, direct endoscopic visualization, ultrasound, or magnetic resonance imaging (MRI).
